(19) United States
(12) Patent Application Publication
(10) Pub. No.: US 2023/0000432 A1
(43) Pub. Date: Jan. 5, 2023

(54) INFORMATION PROCESSING METHOD, INFORMATION PROCESSING DEVICE, AND NON-TRANSITORY COMPUTER READABLE RECORDING MEDIUM STORING INFORMATION PROCESSING PROGRAM

(71) Applicant: Panasonic Intellectual Property Corporation of America, Torrance, CA (US)

(72) Inventors: Yuko KIDA, Osaka (JP); Hiroya TANAKA, Osaka (JP)

(73) Assignee: Panasonic Intellectual Property Corporation of America, Torrance, CA (US)

(21) Appl. No.: 17/943,744

(22) Filed: Sep. 13, 2022

Related U.S. Application Data
(63) Continuation of application No. PCT/JP2021/004704, filed on Feb. 9, 2021.
(60) Provisional application No. 62/990,208, filed on Mar. 16, 2020.

(30) Foreign Application Priority Data
Dec. 21, 2020 (JP) 2020-211533

(51) Int. Cl. A61B 5/00; A61B 5/20

(52) U.S. Cl. CPC A61B 5/4875; A61B 5/4809; A61B 5/207

(57) ABSTRACT

Provided is a life support device including: a sensing information acquisition unit that acquires sensing information output from a sensor that senses a state of a person; a first estimation processor that executes first estimation processing of estimating a state of substance intake of the person based on the sensing information; a second estimation processor that executes second estimation processing of estimating a state of activity of the person, the state being correlated with the state of substance intake, based on the sensing information; a life support information generator that generates life support information on the person based on an execution result of the first estimation processing and an execution result of the second estimation processing; and a life support information output unit that outputs the life support information generated.

### TECHNICAL FIELD

The present disclosure relates to a technique for managing health of a person.

### BACKGROUND ART

In recent years, care management has been introduced as a mechanism for securing integrated provision of a nursing care service suitable for mental and physical conditions of a user, and selection of a service by the user itself, at a job site level. The care management is performed in the order of an intake, an assessment, creation of a care plan, holding of a meeting of persons in charge of service, determination of a care plan, implementation of a service, monitoring, and evaluation.

Multiple assessment items are set for each support content to be provided to the user. A care support specialist (care manager) engaged in care management assesses the multiple assessment items when determining support contents. However, the assessment items are not necessarily provided with information. For example, support contents for enabling water to be taken daily include an assessment item for evaluating whether necessary water is taken. To evaluate this assessment item, monitoring of how much the user takes water daily is required. However, it is difficult to monitor a state of water intake of the user when the user is at home, particularly when the user is alone.

For example, an information processing device disclosed in Patent Literature 1 acquires behavior history information including time information on behavior performed by a user and information indicating a content of the behavior, and estimates a degree of life inactivity indicating a degree of inactivity in life of the user based on the behavior history information. When determining that the degree of life inactivity is inappropriate, the information processing device extracts information on behavior that is likely to be performed by the user, as proposal candidates, from life behavior information including the information on the behavior that is likely to be performed by the user, based on the behavior history information. The information processing device selects behavior that is highly likely to be performed by the user at the time of proposal as a proposal behavior to be proposed from among the proposal candidates. The information processing device generates a proposal message of proposing the proposal behavior to the user, and presents the proposal message to the user.

Unfortunately, the conventional technique described above is less likely to sufficiently support life of a person, and thus further improvement is required.

### CITATION LIST

### Patent Literature

Patent Literature 1: JP 2015-49825 A

### SUMMARY OF INVENTION

The present disclosure is made to solve the above problem, and an object thereof is to provide a technique capable of sufficiently supporting life of a person.

An information processing method according to an aspect of the present disclosure includes, by a computer: acquiring sensing information output from a sensor that senses a state of a person; executing first estimation processing of estimating a state of substance intake of the person based on the sensing information; executing second estimation processing of estimating a state of activity of the person, the state being correlated with the state of substance intake, based on the sensing information; generating life support information on the person based on an execution result of the first estimation processing and an execution result of the second estimation processing; and outputting the life support information generated.

The present disclosure enables life of a person to be sufficiently supported.

### BRIEF DESCRIPTION OF DRAWINGS

FIG. 1 is a block diagram illustrating a configuration of a life support system according to a first embodiment of the present disclosure.

FIG. 2 is a flowchart for illustrating life support processing in a life support device according to the first embodiment of the present disclosure.

FIG. 3 is a flowchart for illustrating life support information generation processing in the first embodiment.

FIG. 4 is a flowchart for illustrating life support information generation processing in a second embodiment.

FIG. 5 is a flowchart for illustrating life support information generation processing in a third embodiment.

FIG. 6 is a flowchart for illustrating life support information generation processing in a modification of the third embodiment.

FIG. 7 is a flowchart for illustrating life support information generation processing in a fourth embodiment.

### DESCRIPTION OF EMBODIMENTS

(Underlying Knowledge of Present Disclosure)

The conventional information processing device described above is configured such that when it is determined that an estimated degree of life inactivity is inappropriate, a proposal behavior to be proposed is selected, and a proposal message of proposing the proposal behavior to a care manager, a family, a nursing care service provider, or the user itself, is presented to the user. In contrast, when it is determined that a degree of life inactivity is appropriate, the conventional information processing device does not present a proposal message to the user. The conventional information processing device estimates behavior contents of the user based on momentum information and position information, but does not estimate a state of substance intake of the user and a state of activity of the user, correlated with the state of substance intake. This causes a difficulty in sufficiently supporting the life of the user.

To solve the above problem, an information processing method according to an aspect of the present disclosure includes, by a computer: acquiring sensing information output from a sensor that senses a state of a person; executing first estimation processing of estimating a state of substance intake of the person based on the sensing information; executing second estimation processing of estimating a state of activity of the person, the state being correlated with the state of substance intake, based on the sensing information; generating life support information on the person based on an execution result of the first estimation processing and an execution result of the second estimation processing; and outputting the life support information generated.

For example, the amount of water taken into a human body correlates with an amount of urine discharged from the human body, and when balance between the amount of water intake and the amount of urine is lost, dehydration may be caused. For this situation, the above configuration generates the life support information on the person based on an estimation result of the state of substance intake of the person and an estimation result of the state of activity of the person, the state being correlated with the state of substance intake, and thus enabling the life of the person to be sufficiently supported.

The information processing method described above may be configured such that, in execution of the first estimation processing, a state of water intake of the person is estimated, in execution of the second estimation processing, a state of excretion of the person is estimated, and in generation of the life support information, the life support information is generated based on balance between the state of water intake and the state of excretion.

This configuration causes the state of water intake of the person and the state of excretion of the person to be estimated, and the life support information to be generated based on the balance between the state of water intake and the state of excretion, so that the person can be supported to take appropriate water.

The information processing method described above may be configured such that, the state of water intake includes an amount of water intake of the person in a predetermined period, the state of excretion includes an amount of urine of the person in the predetermined period, and in generation of the life support information, the life support information indicating that necessary water is not taken is generated when the amount of water intake is less than the amount of urine in comparison between the amount of water intake and the amount of urine in the predetermined period.

This configuration causes the life support information indicating that the necessary water is not taken to be generated when the amount of water intake of the person is smaller than the amount of urine, so that the life of the person can be improved in taking the necessary water.

The information processing method described above may be configured such that, in execution of the first estimation processing, a state of nutrient intake of the person is estimated, in execution of the second estimation processing, a state of nutrient consumption of the person is estimated, and in generation of the life support information, the life support information is generated based on balance between the state of nutrient intake and the state of nutrient consumption.

This configuration causes the state of nutrient intake of the person and the state of nutrient consumption of the person to be estimated, and the life support information to be generated based on the balance between the state of nutrient intake and the state of nutrient consumption, so that the person can be supported to take appropriate nutrient.

The information processing method described above may be configured such that the state of nutrient intake includes the amount of energy intake of the person in a predetermined period, the state of nutrient consumption includes the amount of energy consumption of the person in the predetermined period, in generation of the life support information, the life support information indicating that the amount of meal of the person is insufficient is generated when the amount of energy intake is smaller than the amount of energy consumption in comparison between the amount of energy intake and the amount of energy consumption in the predetermined period, and the life support information indicating that the amount of exercise of the person is insufficient is generated when the amount of energy intake is larger than the amount of energy consumption in the comparison.

This configuration causes the life support information indicating that the amount of meal of the person is insufficient to be generated when the amount of energy intake is smaller than the amount of energy consumption, so that the life of the person can be improved in taking necessary energy. When the amount of energy intake is larger than the amount of energy consumption, the life support information indicating that the amount of exercise of the person is insufficient is generated, and thus the life of the person can be improved in consuming necessary energy.

The information processing method described above may be configured such that, in execution of the first estimation processing, a state of medicine intake of the person is estimated, in execution of the second estimation processing, a state of excretion of the person is estimated, and in generation of the life support information, the life support information is generated based on the state of medicine intake and the state of excretion.

This configuration causes the state of medicine intake of the person and the state of excretion of the person to be estimated, and the life support information to be generated based on the state of medicine intake and the state of excretion, so that the person can be supported to take appropriate medicine.

The information processing method described above may be configured such that the state of medicine intake includes a type, an amount, and a dosage date and time of medicine taken by the person, the state of excretion includes properties, an amount, and an excretion date and time of feces excreted by the person, in generation of the life support information, the life support information prompting consultation with a doctor about medication is generated when it is determined that: the properties of the feces excreted by taking the medicine of the estimated type is not same as the estimated properties of the feces; the amount of the feces excreted by taking the medicine of the estimated type is not same as the estimated amount of the feces; or the feces are not excreted within a predetermined time from the date and time of taking the medicine of the estimated type, and the life support information includes the type, the amount, and the dosage date and time, of the estimated medicine, and the properties, the amount of excretion, and the excretion date and time of the feces.

This configuration causes the life support information prompting consultation with a doctor about medication of the person to be generated when it is determined that: the properties of the feces excreted by taking the medicine of the estimated type is not the same as the estimated properties of the feces; the amount of the feces excreted by taking the medicine of the estimated type is not the same as the estimated amount of the feces; or the feces are not excreted within a predetermined time from the date and time of taking the medicine of the estimated type, so that the consultation with the doctor about the medication of the person can be prompted.

The information processing method described above may be configured such that the state of medicine intake includes a type, an amount, and a dosage date and time of medicine taken by the person, the state of excretion includes an excretion date and time and an excretion frequency of urine excreted by the person, in generation of the life support information, the life support information prompting consultation with a doctor about medication is generated when it is determined that the urine is not excreted within a predetermined time from a date and time when medicine of an estimated type is taken, or when it is determined that an estimated excretion frequency of the urine is higher than a predetermined frequency from the date and time when the medicine of the estimated type is taken, and the life support information includes the type, the amount, and the dosage date and time, of the estimated medicine, and the excretion date and time, and the excretion frequency, of the urine.

This configuration causes the life support information prompting consultation with a doctor about medication of the person to be generated when it is determined that urine is not excreted within a predetermined time from the date and time when the medicine of the estimated type is taken, or when it is determined that the estimated excretion frequency of urine is higher than a predetermined frequency from the date and time when the medicine of the estimated type is taken, so that the consultation with the doctor about the medication of the person can be prompted.

The information processing method described above may be configured such that, in execution of the first estimation processing, a state of medicine intake of the person is estimated, in execution of the second estimation processing, a state of sleep of the person is estimated, and in generation of the life support information, the life support information is generated based on the state of medicine intake and the state of sleep.

This configuration causes the state of medicine intake of the person and the state of sleep of the person to be estimated, and the life support information to be generated based on the state of medicine intake and the state of sleep, so that the person can be supported to take appropriate medicine.

The information processing method described above may be configured such that the state of medicine intake includes a type, an amount, and a dosage date and time of medicine taken by the person, the state of sleep includes sleeping time of the person, in generation of the life support information, the life support information prompting consultation with a doctor about medication is generated when the type of the medicine taken by the person is a sleeping medicine and it is determined that the sleeping time is shorter than a predetermined time after determining whether the sleeping time is shorter than the predetermined time, and the life support information includes the type, the amount, and the dosage date and time, of the estimated medicine, and the sleeping time.

This configuration causes the life support information prompting consultation with a doctor about medication of the person to be generated when it is determined that the type of medicine taken by the person is a sleeping medicine and the sleeping time is shorter than the predetermined time, so that the consultation with the doctor about the medication of the person can be prompted.

An information processing device according to another aspect of the present disclosure includes: an acquisition unit that acquires sensing information output from a sensor that senses a state of a person; a first estimation processing execution unit that executes first estimation processing of estimating a state of substance intake of the person based on the sensing information; a second estimation processing execution unit that executes second estimation processing of estimating a state of activity of the person, the state being correlated with the state of substance intake, based on the sensing information; a generator that generates life support information on the person based on an execution result of the first estimation processing and an execution result of the second estimation processing; and an output unit that outputs the life support information generated.

For example, the amount of water taken into a human body correlates with an amount of urine discharged from the human body, and when balance between the amount of water intake and the amount of urine is lost, dehydration may be caused. For this situation, the above configuration generates the life support information on the person based on an estimation result of the state of substance intake of the person and an estimation result of the state of activity of the person, the state being correlated with the state of substance intake, and thus enabling the life of the person to be sufficiently supported.

A non-transitory computer readable recording medium storing an information processing program according to yet another aspect of the present disclosure causes a computer to function so as to: acquire sensing information output from a sensor that senses a state of a person; execute first estimation processing of estimating a state of substance intake of the person based on the sensing information; execute second estimation processing of estimating a state of activity of the person, the state being correlated with the state of substance intake, based on the sensing information; generate life support information on the person based on an execution result of the first estimation processing and an execution result of the second estimation processing; and output the life support information generated.

For example, the amount of water taken into a human body correlates with an amount of urine discharged from the human body, and when balance between the amount of water intake and the amount of urine is lost, dehydration may be caused. For this situation, the above configuration generates the life support information on the person based on an estimation result of the state of substance intake of the person and an estimation result of the state of activity of the person, the state being correlated with the state of substance intake, and thus enabling the life of the person to be sufficiently supported.

Hereinafter, embodiments of the present disclosure will be described with reference to the accompanying drawings. The following embodiments are merely examples embodying the present disclosure, and do not limit the technical scope of the present disclosure.

### First Embodiment

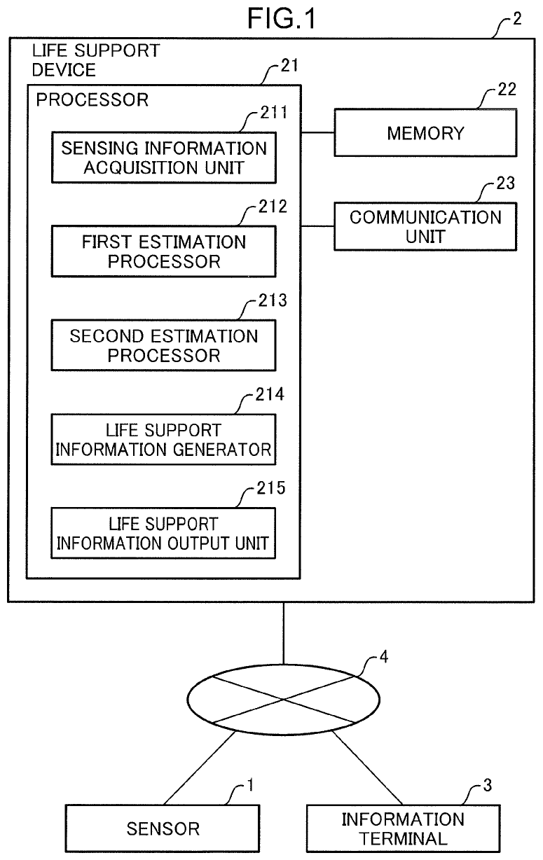

FIG. 1 is a block diagram illustrating a configuration of a life support system according to a first embodiment of the present disclosure.

The life support system illustrated in FIG. 1 includes a sensor 1, a life support device 2, and an information terminal 3.

The sensor 1 senses a state of a person. The sensor 1 transmits sensing information to the life support device 2.

The sensor 1 is provided in a cup used by the person, and includes a sensor that measures the amount of beverage poured into the cup as the amount of water intake of the person, for example. The sensor 1 transmits sensing information indicating the amount of water intake of the person to the life support device 2. The sensor 1 transmits the sensing information to the life support device 2 at timing when the amount of water intake is measured.

The sensor 1 includes a sensor that is provided in a toilet bowl of a toilet to measure a concentration of an ammonia component in a space in the toilet bowl, for example. The sensor 1 transmits sensing information indicating the concentration of the ammonia component in the space in the toilet bowl to the life support device 2. The sensor 1 transmits the sensing information to the life support device 2 at timing when the concentration of the ammonia component in the space in the toilet bowl is measured.

The sensor 1 may further include an input device that receives the fact that the person has taken water, the fact being input by the person itself, for example. When having taken water, the person presses a button provided in the input device. When the button is pressed, the input device may transmit sensing information indicating that water is taken by the person to the life support device 2. The input device may receive the amount of water intake, which is input by the person, and transmit sensing information indicating the amount of water intake to the life support device 2.

The life support device 2 is a server, for example. The life support device 2 is communicably connected to the sensor 1 via a network 4. The network 4 is the Internet, for example. The life support device 2 is communicably connected to the information terminal 3 via the network 4.

The life support device 2 includes a processor 21, a memory 22, and a communication unit 23.

The communication unit 23 receives the sensing information transmitted by the sensor 1.

The memory 22 is a storage device capable of storing various types of information, such as a random access memory (RAM), a hard disk drive (HDD), a solid state drive (SSD), or a flash memory. The memory 22 stores the sensing information transmitted by the sensor 1.

The processor 21 is a central processing unit (CPU), for example. The processor 21 serves as a sensing information acquisition unit 211, a first estimation processor 212, a second estimation processor 213, a life support information generator 214, and a life support information output unit 215.

The sensing information acquisition unit 211 acquires sensing information output from the sensor 1 that senses a state of the person. The sensing information acquisition unit 211 acquires the sensing information from the memory 22. The sensing information acquisition unit 211 reads out the sensing information stored in the memory 22.

The first estimation processor 212 executes the first estimation processing of estimating a state of substance intake of the person based on the sensing information. The first estimation processor 212 estimates a state of water intake of the person in the first estimation processing. The state of water intake includes the amount of water taken by the person in a predetermined period. The predetermined period is one day, for example. The first estimation processor 212 estimates the amount of water intake of the person in the predetermined period by summing the amounts of water intake in the predetermined period acquired as the sensing information.

Although in the first embodiment, the first estimation processor 212 estimates the amount of water intake based on the sensing information from a sensor that measures the amount of beverage poured into a cup as the amount of water intake of the person, the present disclosure is not particularly limited thereto. For example, the first estimation processor 212 may recognize a behavior of a person drinking a beverage from an image acquired from a camera that captures an image of the person. Then, the first estimation processor 212 may measure time during which the person drinks the beverage and estimate the amount of water intake from the time measured.

The second estimation processor 213 executes the second estimation processing of estimating a state of activity of the person, the state being correlated with the state of substance intake, based on the sensing information. The second estimation processor 213 estimates a state of excretion of the person in the second estimation processing. The state of excretion includes an amount of urine of the person in a predetermined period. The predetermined period is one day, for example. The second estimation processor 213 sums time during which the concentration of the ammonia component in the predetermined period acquired as the sensing information continuously exceeds a threshold. That is, when the person discharges urine, the concentration of the ammonia component in the toilet bowl increases. Thus, time during which the person urinates can be calculated by calculating time from when the concentration of the ammonia component in the toilet bowl exceeds the threshold until when the concentration falls below the threshold. The second estimation processor 213 estimates an amount of urine of the person in a predetermined period (e.g., one day) by multiplying the summed time by the amount of urine discharged per unit time (e.g., one second). The amount of urine discharged per unit time is preliminarily stored in the memory 22.

Although in the first embodiment, the second estimation processor 213 estimates the amount of urine of the person based on the sensing information from the sensor that measures the concentration of the ammonia component in the space in the toilet bowl, the present disclosure is not particularly limited thereto. For example, the second estimation processor 213 may determine whether the person urinates based on sound information from a microphone that collects sounds in the space in the toilet bowl. The second estimation processor 213 may estimate an amount of urine of the person based on time during which it is continuously determined that the person urinates, and the amount of urine discharged per unit time (e.g., one second).

The sensor 1 is preferably a non-contact sensor to acquire sensing information. This enables estimating the state of substance intake and the state of activity of the person without imposing a burden on the person. The sensor 1 may be a contact-type sensor such as a wearable sensor, for example.

At least one of the first estimation processing and the second estimation processing may be performed based on observation information acquired by a care service provider, a family, or the like.

The life support information generator 214 generates life support information on the person based on an execution result of the first estimation processing and an execution result of the second estimation processing. The life support information generator 214 generates the life support information based on balance between the state of water intake and the state of excretion. The life support information generator 214 compares the amount of water intake with an amount of urine in a predetermined period. When the amount of water intake is smaller than the amount of urine, the life support information generator 214 generates life support information indicating that necessary water is not taken. In contrast, when the amount of water intake is equal to or more than the amount of urine, the life support information generator 214 generates life support information indicating that necessary water has be taken.

The life support information output unit 215 outputs the life support information generated by the life support information generator 214. For example, the life support information output unit 215 outputs the life support information indicating whether necessary water is taken. The life support information output unit 215 transmits the life support information for supporting a life of the person to the information terminal 3 using the communication unit 23.

The communication unit 23 transmits the life support information output by the life support information output unit 215 to the information terminal 3.

The information terminal 3 is a smartphone, a tablet computer, or a personal computer, for example. The information terminal 3 receives the life support information transmitted by the life support device 2. The information terminal 3 presents the life support information received. For example, the information terminal 3 displays the life support information received.

The information terminal 3 is used by a care manager, for example. The care manager creates a care plan for a person requiring care or a person requiring support by using life support information on a person in a state having been sensed (e.g., the person requiring care or the person requiring support). That is, one of the assessment items is for selecting whether necessary water is taken. The care manager creates a care plan for the person requiring care or the person requiring support by using the life support information indicating whether necessary water is taken.

Although in the first embodiment, the life support information is transmitted to the information terminal 3 used by the care manager, the present disclosure is not particularly limited thereto. The life support information may be transmitted to the info nation terminal 3 used by the person in the state having been sensed or a caregiver of the person. When necessary water is not taken, dehydration may occur. Thus, when receiving the life support information indicating that the necessary water is not taken, the information terminal 3 may present a message prompting the person to take water.

The life support information may be transmitted to the information terminal 3 used by a service provider that provides a service to the person.

Subsequently, life support processing in the life support device 2 according to the first embodiment of the present disclosure will be described.

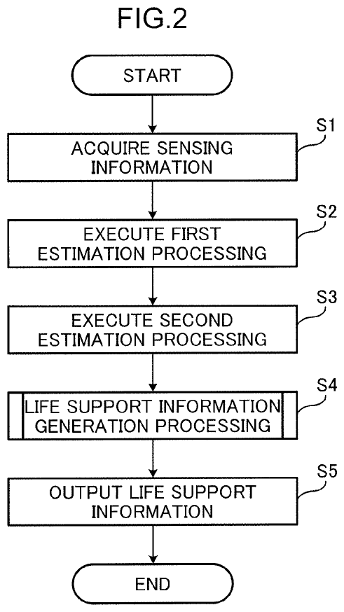

FIG. 2 is a flowchart for illustrating the life support processing in the life support device 2 according to the first embodiment of the present disclosure.

In step S1, the sensing information acquisition unit 211 first acquires sensing information measured by the sensor 1 from the memory 22. The sensing information is the amount of water intake and an amount of urine, for example.

For example, the life support processing illustrated in FIG. 2 is performed once a day. The sensing information acquisition unit 211 acquires sensing information for one day at 0:00 AM, for example. Time at which the sensing information is acquired is not limited to 0:00 AM. The life support processing illustrated in FIG. 2 is not limited to once a day, and may be performed multiple times a day, once a week, or every predetermined period.

In step S2, the first estimation processor 212 subsequently executes the first estimation processing of estimating the state of substance intake of the person based on the sensing information acquired by the sensing information acquisition unit 211. Here, the first estimation processor 212 estimates the amount of water intake of the person in a predetermined period.

In step S3, the second estimation processor 213 subsequently executes the second estimation processing of estimating the state of activity of the person, the state being correlated with the state of substance intake, based on the sensing information acquired by the sensing information acquisition unit 211. Here, the second estimation processor 213 estimates an amount of urine of the person in a predetermined period.

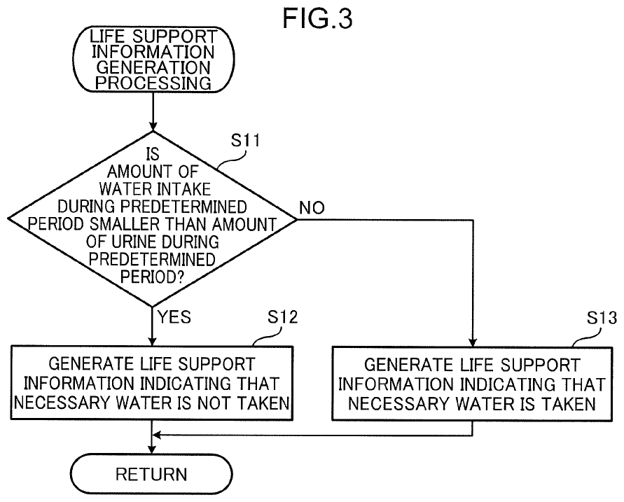

In step S4, the life support information generator 214 subsequently executes life support information generation processing of generating life support information on the person based on an execution result of the first estimation processing and an execution result of the second estimation processing. The life support information generation processing will be described later with reference to FIG. 3.

In step S5, the life support information output unit 215 subsequently outputs the life support information generated by the life support information generator 214. For example, the life support information output unit 215 transmits the life support information indicating whether necessary water is taken to the information terminal 3 using the communication unit 23.

Subsequently. the life support information generation processing in step S4 of FIG. 2 will be described.

FIG. 3 is a flowchart for illustrating the life support information generation processing in the first embodiment.

In step S11, the life support information generator 214 first determines whether the amount of water intake during the predetermined period estimated by the first estimation processor 212 is smaller than the amount of urine during the predetermined period estimated by the second estimation processor 213.

Here, when determining that the amount of water intake during the predetermined period is smaller than the amount of urine during the predetermined period (YES in step S11). the life support information generator 214 generates life support information indicating that necessary water is not taken, in step S12.

In contrast, when determining that the amount of water intake during the predetermined period is equal to or more than the amount of urine during the predetermined period (NO in step S11), the life support information generator 214 generates life support information indicating that necessary water is taken. in step S13.

As described above, the amount of water taken into a human body correlates with an amount of urine discharged from the human body, for example, and when balance between the amount of water intake and the amount of urine is lost, dehydration may be caused. For this situation, the first embodiment causes the life support information on the person to be generated based on an estimation result of the state of substance intake of the person and an estimation result of the state of activity of the person, the state being correlated with the state of substance intake, and thus enabling the life of the person to be sufficiently supported.

### Second Embodiment

The life support information generator 214 in the first embodiment determines whether the amount of water intake during the predetermined period estimated by the first estimation processor 212 is smaller than the amount of urine during the predetermined period estimated by the second estimation processor 213. In contrast, the life support information generator 214 in a second embodiment determines whether the amount of energy intake during a predetermined period estimated by the first estimation processor 212 is smaller than the amount of energy consumption during the predetermined period estimated by the second estimation processor 213.

A life support system in the second embodiment is identical in configuration to the life support system in the first embodiment. Thus, the configuration of the life support system according to the second embodiment will be described below with reference to FIG. 1.

The sensor 1 in the second embodiment includes a camera that is provided above a table at which a person has a meal and that captures an image from above of the table on which the meal is placed. The sensor 1 transmits sensing information indicating the image, in which the meal of the person is captured, to the life support device 2.

The sensor 1 according to the second embodiment includes at least one of a plurality of motion sensors provided in a house and a plurality of opening-closing sensors provided on doors of respective rooms in the house. The sensor 1 transmits sensing information to the life support device 2, the sensing information indicating a position where the person is detected or a position of an opened or closed door.

The sensor 1 may further include an input device that receives the fact that the person has taken a meal, the fact being input by the person itself, for example. When having taken a meal, the person presses a button provided in the input device. When the button is pressed, the input device may transmit sensing information indicating that the meal is taken by the person to the life support device 2. The input device may receive meal contents, which are input by the person, and transmit sensing information indicating the meal contents to the life support device 2.

The sensor 1 may further include a home appliance that receives an operation of the person and operates in accordance with the operation received. The home appliance may transmit operation log information as sensing information to the life support device 2. The life support device 2 can estimate a state of activity of the person based on the operation log information from the home appliance.

The first estimation processor 212 in the second embodiment estimates a state of nutrient intake of the person in first estimation processing. The state of nutrient intake includes the amount of energy intake of the person in a predetermined period. The predetermined period is one day, for example. A unit of the amount of energy intake is a calorie. The first estimation processor 212 recognizes contents of a meal from an image acquired as sensing information by using an image recognition processing technique. The first estimation processor 212 estimates the amount of energy intake from the contents of the meal recognized. The memory 22 preliminarily stores a table in which contents of a meal are associated with the amount of energy intake. Then, the first estimation processor 212 estimates the amount of energy intake of the person in the predetermined period by summing the amounts of energy intake in the predetermined period.

The second estimation processor 213 in the second embodiment estimates a state of nutrient consumption of the person in second estimation processing. The state of nutrient consumption includes the amount of energy consumption of the person in a predetermined period. The predetermined period is one day, for example. A unit of the amount of energy consumption is a calorie. The second estimation processor 213 estimates a travel distance of the person in the predetermined period from identification information acquired by a motion sensor or an opening-closing sensor as sensing information. The memory 22 preliminarily stores a table in which a combination of two identification information pieces acquired by one of multiple motion sensors and one of multiple opening-closing sensors provided in a house of the person is associated with a distance between the two sensors. The memory 22 also preliminarily stores a table in which a travel distance is associated with the amount of energy consumption. The second estimation processor 213 reads out a distance associated with a combination of identification information received this time and identification information received last time from the memory 22 as the travel distance of the person. The second estimation processor 213 sums travel distances in the predetermined period, and reads out the amount of energy consumption associated with the travel distances summed. As a result, the second estimation processor 213 estimates the amount of energy consumption of the person in the predetermined period.

For example, detection of an opening-closing sensor at a door of a toilet after detection of an opening-closing sensor at a door of a living room enables determining that the person has traveled from the living room to the toilet. Thus, a distance between the opening-closing sensor at the door of the living room and the opening-closing sensor at the door of the toilet is estimated as the travel distance of the person.

The life support information generator 214 in the second embodiment generates life support information based on balance between the state of nutrient intake and the state of nutrient consumption. The life support information generator 214 compares the amount of energy intake and the amount of energy consumption in a predetermined period. When the amount of energy intake is smaller than the amount of energy consumption, the life support information generator 214 generates the life support information indicating that the amount of meal of the person is insufficient. In contrast, when the amount of energy intake is larger than the amount of energy consumption, the life support information generator 214 generates the life support information indicating that the amount of exercise of the person is insufficient.

For example, the life support information output unit 215 outputs the life support information indicating that the amount of meal of the person is insufficient or the life support information indicating that the amount of exercise of the person is insufficient. The life support information output unit 215 transmits the life support information for supporting a life of the person to the information terminal 3 using the communication unit 23.

For example, when the information terminal 3 receives the life support information indicating that the amount of meal of the person is insufficient, a care manager creates a care plan for improving a meal support system. When the information terminal 3 receives the life support information indicating that the amount of exercise of the person is insufficient, for example, the care manager creates a care plan for improving an exercise support system.

Although in the second embodiment, the sensor 1 includes the camera that captures an image from above of a table on which a meal is placed, the present disclosure is not particularly limited thereto. The sensor 1 may include a camera that is provided in a refrigerator to capture an image of foodstuffs in the refrigerator. In this case, the first estimation processor 212 may recognize the foodstuffs in the refrigerator and recognize foodstuffs taken out from the refrigerator from the image acquired as sensing information by using the image recognition processing technique. The first estimation processor 212 may estimate a cooked dish from the foodstuffs taken out from the refrigerator and estimate the amount of energy intake from a type of the dish estimated. The memory 22 preliminarily stores a table in which multiple foodstuffs and types of dishes cooked using the corresponding multiple foodstuffs are associated with each other. The memory 22 also preliminarily stores a table in which a type of dish is associated with the amount of energy intake. Then, the first estimation processor 212 may estimate the amount of energy intake of the person in a predetermined period by summing the amounts of energy intake in the predetermined period.

Although in the second embodiment, the sensor 1 includes a motion sensor or a door opening-closing sensor, the present disclosure is not particularly limited thereto. The sensor 1 may include an activity meter that measures the amount of activity of a person. The activity meter is attached to a person. The amount of activity is the number of steps of a person, for example. The memory 22 may preliminarily store a table in which the number of steps is associated with the amount of energy consumption. In this case, the second estimation processor 213 may read out the amount of energy consumption associated with the number of steps in the predetermined period from the memory 22.

The sensor 1 may further include a camera that captures an image of a person in the house. The second estimation processor 213 may recognize life behavior of the person from the image captured by the camera. The life behavior is cleaning, washing, cooking, bathing, and the like, for example. The memory 22 may preliminarily store a table in which the life behavior is associated with the amount of energy consumption. The second estimation processor 213 may read out the amount of energy consumption associated with the recognized life behavior from the memory 22, and estimate a total amount of energy consumption read out within the predetermined period as the amount of energy consumption in the predetermined period.

Subsequently, life support information generation processing in the second embodiment will be described. Life support processing other than the life support information generation processing in the second embodiment is the same as the life support processing in the first embodiment illustrated in FIG. 2.

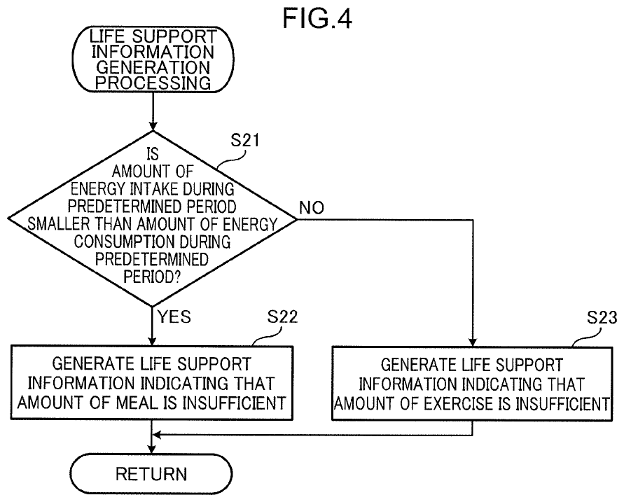

FIG. 4 is a flowchart for illustrating the life support information generation processing in the second embodiment.

In step S21, the life support information generator 214 first determines whether the amount of energy intake during a predetermined period estimated by the first estimation processor 212 is smaller than the amount of energy consumption during the predetermined period estimated by the second estimation processor 213.

Here. when determining that the amount of energy intake during a predetermined period is smaller than the amount of energy consumption during the predetermined period (YES in step S21), the life support information generator 214 generates life support information indicating that the amount of meal of the person is insufficient, in step S22.

In contrast, when determining that the amount of energy intake during the predetermined period is equal to or more than the amount of energy consumption during the predetermined period (NO in step S21), the life support information generator 214 generates life support information indicating that the amount of exercise of the person is insufficient, in step S23.

As described above, the second embodiment causes the state of nutrient intake of the person and the state of nutrient consumption of the person to be estimated, and the life support information to be generated based on the balance between the state of nutrient intake and the state of nutrient consumption, so that the person can be supported to take appropriate nutrient.

### Third Embodiment

The life support information generator 214 in a third embodiment determines whether properties of feces excreted by taking a medicine of a type estimated by the first estimation processor 212 are the same as properties of feces estimated by the second estimation processor 213.

A life support system in the third embodiment is identical in configuration to the life support system in the first embodiment. Thus, the configuration of the life support system according to the third embodiment will be described below with reference to FIG. 1.

The sensor 1 in the third embodiment includes a dosage support device that notifies a scheduled dosage time with an image and/or a voice and notifies a type, the amount, and a dosage date and time, of medicine taken out by a person. The sensor 1 transmits sensing information to the life support device 2, the sensing information indicating the type, the amount, and the dosage date and time, of the medicine taken out by the person.

The sensor 1 in the third embodiment also includes a camera provided in a toilet bowl of a toilet. The sensor 1 transmits sensing information indicating an image obtained by capturing feces excreted in the toilet bowl to the life support device 2.

The sensor 1 may further include an input device that receives the fact that the person has taken a medicine, the fact being input by the person itself, for example. When having taken a medicine, the person presses a button provided in the input device. When the button is pressed, the input device may transmit sensing information indicating that the medicine is taken by the person to the life support device 2. The input device may receive the type, the amount, and the dosage date and time, of the medicine taken, which are input by the person, and transmit sensing information to the life support device 2, the sensing information indicating the type, the amount, and the dosage date and time, of the medicine.

The sensor 1 may further include an input device that receives the fact that the person has defecated or urinated, the fact being input by the person itself, for example. When having defecated or urinated, the person presses a button provided in the input device. When the button is pressed, the input device may transmit sensing information indicating that the person has defecated or urinated to the life support device 2. The input device may receive properties, the amount, and an excretion date and time, of feces excreted by the person, and transmit sensing information to the life support device 2, the sensing information indicating the properties, the amount, and the excretion date and time, of the feces. The input device may also receive an excretion date and time, and an excretion frequency of urine excreted by a person, which are input by the person, and transmit sensing information indicating the excretion date and time, and the excretion frequency of urine, to the life support device 2.

The first estimation processor 212 in the third embodiment estimates a state of medicine intake of the person in first estimation processing. The state of medicine intake includes a type, the amount, and a dosage date and time, of medicine taken by the person in a predetermined period. The predetermined period is one day, for example. The first estimation processor 212 estimates a type, the amount, and a dosage date and time, of medicine taken by the person, from the type, the amount, and the dosage date and time of the medicine acquired as the sensing information.

The second estimation processor 213 in the third embodiment estimates a state of excretion of the person in second estimation processing. The state of excretion includes properties, the amount, and an excretion date and time, of feces excreted by the person in a predetermined period. The predetermined period is one day, for example. The second estimation processor 213 estimates the properties, the amount, and the excretion date and time, of the feces excreted by the person in the predetermined period from an image acquired as sensing information using the image recognition processing technique. Examples of an index for classifying properties of feces include a Bristol stool scale. According to the Bristol stool scale, the properties of feces is classified into ball-like feces, hard feces, slightly hard feces, ordinary feces, slightly soft feces, muddy feces, and watery feces. When an image obtained by capturing feces is recognized, properties of the feces can be identified. The properties of feces are not limited to the seven types above, and may be more than seven types or less than seven types.

The life support information generator 214 in the third embodiment generates life support information based on the state of medicine intake and the state of excretion. The life support information generator 214 determines whether properties of feces excreted by taking the medicine of the estimated type is the same as the estimated properties of feces. The memory 22 preliminarily stores a table in which the type of medicine is associated with the properties of feces excreted by taking the medicine of the type. When determining that the properties of feces excreted by taking the medicine of the estimated type is not the same as the estimated properties of feces, the life support information generator 214 generates life support information prompting consultation with a doctor about medication. At this time, the life support information generator 214 generates the life support information including an estimated type, the amount, and a dosage date and time, of medicine, and properties of feces, the amount of excretion, and an excretion date and time.

The life support information generator 214 determines whether the amount of feces excreted by taking the medicine of the estimated type is the same as the estimated amount of feces. The memory 22 preliminarily stores a table in which the type of medicine is associated with the amount of feces excreted by taking the medicine of the type. When determining that the amount of feces excreted by taking the medicine of the estimated type is not the same as the estimated amount of feces, the life support information generator 214 generates the life support information prompting consultation with a doctor about medication. At this time, the life support information generator 214 generates the life support information including an estimated type, the amount, and a dosage date and time, of medicine, and properties of feces, the amount of excretion, and an excretion date and time.

The life support information generator 214 also determines whether feces are excreted within a predetermined time from the date and time when the medicine of the estimated type is taken. The memory 22 preliminarily stores a table in which a type of medicine is associated with a predetermined time from when the medicine of the type is taken until feces are excreted. When determining that feces are not excreted within the predetermined time from the date and time when the medicine of the estimated type is taken, the life support information generator 214 generates life support information prompting consultation with a doctor about medication. At this time, the life support information generator 214 generates the life support information including an estimated type, the amount, and a dosage date and time, of medicine, and properties of feces, the amount of excretion, and an excretion date and time.

In contrast, the life support information generator 214 generates life support information indicating that the person is correctly taking the medicine when determining that: the properties of feces excreted by taking the medicine of the estimated type is the same as the estimated properties of feces; the amount of feces excreted by taking the medicine of the estimated type is the same as the estimated amount of feces; and feces are excreted within the predetermined time from the date and time when the medicine of the estimated type is taken. At this time, the life support information generator 214 generates the life support information including an estimated type, the amount, and a dosage date and time, of medicine, and properties of feces, the amount of excretion, and an excretion date and time.

For example, when the medicine of the estimated type is a laxative, properties of feces excreted by taking the laxative is any one of slightly soft feces, muddy feces, and watery feces. At this time, when determining that the estimated properties of the feces are not any of the slightly soft feces, the muddy feces, and the watery feces, the life support information generator 214 generates life support information prompting consultation with a doctor about medication.

For example, the life support information output unit 215 outputs life support information indicating that the person is correctly taking the medicine or life support information prompting consultation with a doctor about medication. The life support information output unit 215 transmits the life support information for supporting a life of the person to the information terminal 3 using the communication unit 23.

For example, when the information terminal 3 receives the life support information prompting consultation with a doctor about medication, a care manager presents the doctor with the type, the amount, the dosage date and time, of the estimated medicine, and the properties of feces, the amount of excretion, and the excretion date and time. The life support information includes the type, the amount, the dosage date and time, of the estimated medicine, and the properties of feces, the amount of excretion, and the excretion date and time. The doctor can determine whether the prescribed medicine is suitable for the person by checking the type, the amount, the dosage date and time, of the medicine presented, and the properties of feces, the amount of excretion, and the excretion date and time. In contrast, when the information terminal 3 receives the life support information indicating that the person is correctly taking the medicine, for example, the care manager may or may not present the doctor with the type, the amount, the dosage date and time, of the estimated medicine, and the properties of feces, the amount of excretion, and the excretion date and time.

Subsequently, life support information generation processing in the third embodiment will be described. Life support processing other than the life support information generation processing in the third embodiment is the same as the life support processing in the first embodiment illustrated in FIG. 2.

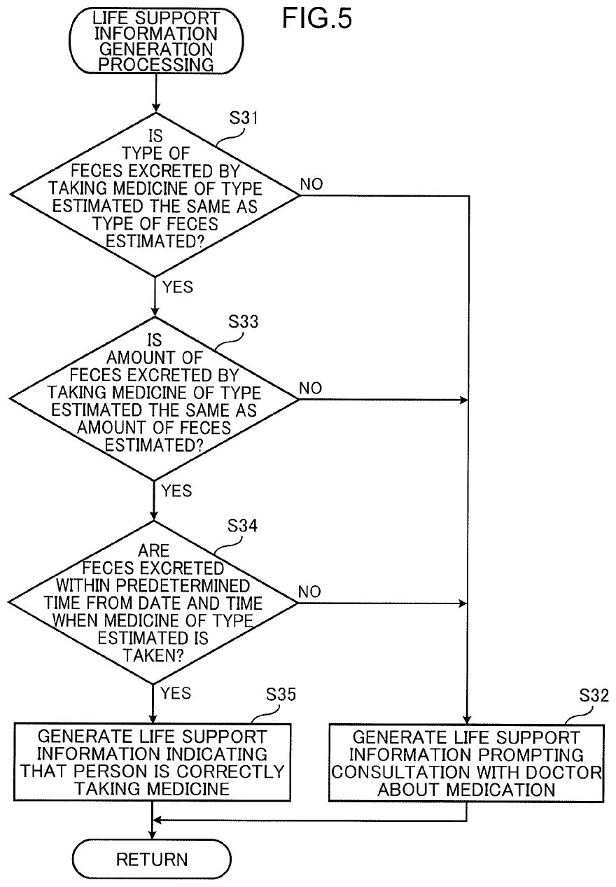

FIG. 5 is a flowchart for illustrating the life support information generation processing in the third embodiment.

In step S31, the life support information generator 214 first determines whether properties of feces excreted by taking the medicine of the type estimated by the first estimation processor 212 are the same as properties of feces estimated by the second estimation processor 213.

Here, when determining that the properties of the feces excreted by taking the medicine of the estimated type is not the same as the estimated properties of the feces (NO in step S31), the life support information generator 214 generates the life support information prompting consultation with a doctor about medication, in step S32.

In contrast, when determining that the properties of the feces excreted by taking the medicine of the estimated type is the same as the estimated properties of the feces (YES in step S31), the life support information generator 214 determines whether the amount of feces excreted by taking the medicine of the type estimated by the first estimation processor 212 is the same as the amount of feces estimated by the second estimation processor 213, in step S33. Here, when it is determined that the amount of feces excreted by taking the medicine of the estimated type is not the same as the estimated amount of feces (NO in step S33), the processing proceeds to step S32.

In contrast, when determining that the amount of feces excreted by taking the medicine of the estimated type is the same as the estimated amount of feces (YES in step S33), the life support information generator 214 determines whether feces are excreted within a predetermined time from the date and time when the medicine of the type estimated by the first estimation processor 212 is taken, in step S34. Here, when it is determined that the feces are not excreted within the predetermined time from the date and time when the medicine of the estimated type is taken (NO in step S34), the processing proceeds to step S32.

In contrast, when determining that the feces are excreted within the predetermined time from the date and time when the medicine of the estimated type is taken (YES in step S34), the life support information generator 214 generates the life support information indicating that the person is correctly taking the medicine, in step S35.

As described above, the third embodiment causes the state of medicine intake of the person and the state of excretion of the person to be estimated, and the life support information to be generated based on the state of medicine intake and the state of excretion, so that the person can be supported to take appropriate medicine.

The life support information prompting consultation with a doctor about medication of the person is generated when it is determined that: the properties of the feces excreted by taking the medicine of the estimated type is not the same as the estimated properties of the feces; the amount of the feces excreted by taking the medicine of the estimated type is not the same as the estimated amount of the feces; or the feces are not excreted within a predetermined time from the date and time when the medicine of the estimated type is taken, so that the consultation with the doctor about the medication of the person can be prompted.

The processing in step S31, step S33, and step S34 may be individually performed. That is, when determining that the properties of the feces excreted by taking the medicine of the estimated type is the same as the estimated properties of the feces (YES in step S31), the life support information generator 214 may generate the life support information indicating that the person is correctly taking the medicine, in step S35, without performing the processing in steps S33 and S34. Alternatively, the processing in step S33 may be performed without performing the processing in step S31. In this case, when determining that the amount of the feces excreted by taking the medicine of the estimated type is the same as the estimated amount of the feces (YES in step S33), the life support information generator 214 may generate the life support information indicating that the person is correctly taking the medicine, in step S35, without performing the processing in step S34. Alternatively, the processing in step S34 may be performed without performing the processing in steps S31 and S33.

Subsequently, a modification of the third embodiment will be described.

The first estimation processor 212 in the modification of the third embodiment estimates a state of medicine intake of the person in the first estimation processing. The state of medicine intake includes a type, the amount, and a dosage date and time, of medicine taken by the person in a predetermined period. The predetermined period is one day, for example. The first estimation processor 212 estimates a type, the amount, and a dosage date and time, of medicine taken by the person, from the type, the amount, and the dosage date and time of the medicine acquired as the sensing information.

The second estimation processor 213 in the modification of the third embodiment estimates a state of excretion of the person in the second estimation processing. The state of excretion includes an excretion date and time and an excretion frequency of urine excreted by the person in a predetermined period. The predetermined period is one day, for example. The second estimation processor 213 estimates a date and time when a concentration of an ammonia component in the predetermined period acquired as sensing information continuously exceeds a threshold, as the excretion date and time of urine. The second estimation processor 213 also estimates a frequency in which the concentration of the ammonia component in the predetermined period acquired as the sensing information continuously exceeds the threshold, as the excretion frequency of urine.

The life support information generator 214 in the modification of the third embodiment generates life support information based on the state of medicine intake and the state of excretion. The life support information generator 214 also determines whether urine is excreted within a predetermined time from a date and time when a medicine of an estimated type is taken. The memory 22 preliminarily stores a table in which a type of medicine is associated with a predetermined time from when the medicine of the type is taken until urine is excreted. When determining that urine is not excreted within the predetermined time from the date and time when the medicine of the estimated type is taken, the life support information generator 214 generates life support information prompting consultation with a doctor about medication. At this time, the life support information generator 214 generates the life support information including an estimated type, the amount, and a dosage date and time, of medicine, and an excretion date and time, and excretion frequency, of urine.

The life support information generator 214 in the modification of the third embodiment also determines whether an estimated excretion frequency of urine is higher than a predetermined frequency from the date and time when the medicine of the estimated type is taken. The memory 22 preliminarily stores a table in which a type of medicine is associated with a predetermined frequency at which urine is excreted after the medicine of the type is taken. When determining that the estimated excretion frequency of urine is higher than the predetermined frequency from the date and time when the medicine of the estimated type is taken, the life support information generator 214 generates the life support information prompting consultation with a doctor about medication. At this time, the life support information generator 214 generates the life support information including an estimated type, the amount, and a dosage date and time, of medicine, and an excretion date and time, and excretion frequency, of urine.

In contrast, when determining not only that urine is excreted within the predetermined time from the date and time when the medicine of the estimated type is taken, but also that the estimated excretion frequency of urine is equal to or less than the predetermined frequency from the date and time when the medicine of the estimated type is taken, the life support information generator 214 generates life support information indicating that the person is correctly taking the medicine. At this time, the life support information generator 214 generates the life support information including an estimated type, the amount, and a dosage date and time, of medicine, and an excretion date and time, and excretion frequency, of urine.

Users with diseases of the circulatory system often take diuretics. However, elderly people may suffer from frequent urination, and thus it is said that control of medicine is difficult. Thus, a dosage of medicine (e.g., a diuretic), an excretion date and time, and an excretion frequency (especially, nocturnal excretion frequency) are important information for supporting life of the users.

Subsequently, life support information generation processing in the modification of the third embodiment will be described. Life support processing other than the life support infatuation generation processing in the modification of the third embodiment is the same as the life support processing in the first embodiment illustrated in FIG. 2.

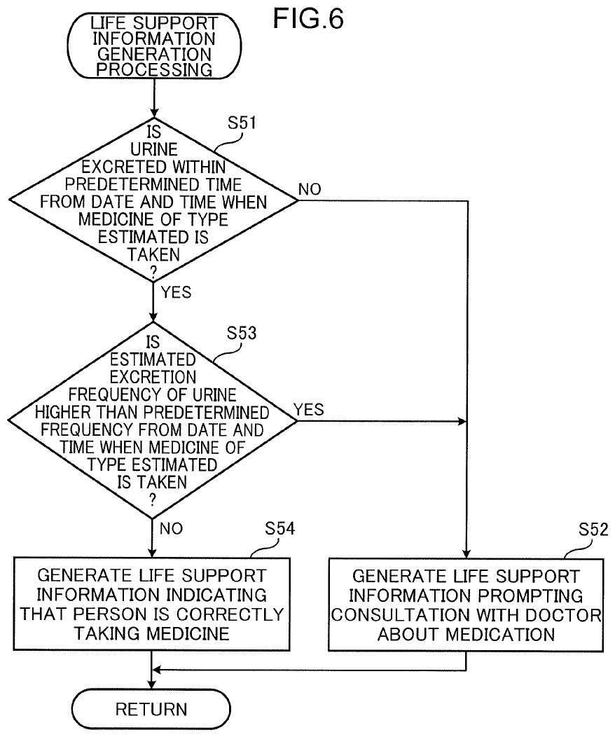

FIG. 6 is a flowchart for the illustrating life support information generation processing in the modification of the third embodiment.

In step S51, the life support information generator 214 first determines whether urine is excreted within the predetermined time from the date and time when the medicine of the type estimated by the first estimation processor 212 is taken. Here, when determining that urine is not excreted within the predetermined time from the date and time when the medicine of the estimated type is taken (NO in step S51), the life support information generator 214 generates the life support information prompting consultation with a doctor about medication, in step S52.

In contrast, when determining that urine is excreted within the predetermined time from the date and time when the medicine of the estimated type is taken (YES in step S51), the life support information generator 214 determines whether the excretion frequency of urine, estimated by the second estimation processor 213, is higher than the predetermined frequency from the date and time when the medicine of the type estimated by the first estimation processor 212 is taken, in step S53.

Here, when it is determined that the estimated excretion frequency of urine is higher than the predetermined frequency from the date and time when the medicine of the estimated type is taken (YES in step S53), the processing proceeds to step S52.

In contrast, when determining that the estimated excretion frequency of urine is equal to or less than the predetermined frequency from the date and time when the medicine of the estimated type is taken (NO in step S53), the life support information generator 214 generates the life support information indicating that the person is correctly taking the medicine, in step S54.

As described above, the life support information prompting consultation with a doctor about medication of the person is generated when it is determined that urine is not excreted within a predetermined time from the date and time when the medicine of the estimated type is taken, or when it is determined that the estimated excretion frequency of urine is higher than a predetermined frequency from the date and time when the medicine of the estimated type is taken, so that the consultation with the doctor about the medication of the person can be prompted.

The processing in steps S51 and S53 may be individually performed. That is, when determining that urine is excreted within the predetermined time from the date and time when the medicine of the estimated type is taken (YES in step S51), the life support information generator 214 generates the life support information indicating that the person is correctly taking the medicine, in step S54, without performing the processing in step S53. Alternatively, the processing in step S53 may be performed without performing the processing in step S51.

### Fourth Embodiment

When a type of medicine taken by the person is a sleeping medicine, a fife support information generator 214 in a fourth embodiment determines whether sleeping time is shorter than a predetermined time.

A life support system in the fourth embodiment is identical in configuration to the life support system in the first embodiment. Thus, the configuration of the life support system according to the fourth embodiment will be described below with reference to FIG. 1.

The sensor 1 in the fourth embodiment includes a dosage support device that notifies scheduled dosage time with an image and/or a voice and notifies a type of medicine taken out by a person. The sensor 1 transmits sensing information to the life support device 2, the sensing information indicating the type of the medicine taken out by the person.

The sensor 1 in the fourth embodiment is a piezoelectric sensor of a sheet-type installed under a bed mattress on which the person sleeps, and acquires biological information such as a heart rate, a respiratory rate, and the amount of body motion, of the person. The sensor 1 periodically (e.g., every 10 minutes) acquires biological information on the person, and transmits sensing information indicating the acquired biological information to the life support device 2. The sensor 1 is not limited to the piezoelectric sensor, and may be a sensor that acquires biological information by another method, such as a wearable sensor of a wristband-type or a radio wave sensor.

The first estimation processor 212 in the fourth embodiment estimates a state of medicine intake of the person in first estimation processing. The state of medicine intake includes a type of medicine taken by the person in a predetermined period. The predetermined period is one day, for example. The first estimation processor 212 estimates a type of medicine taken by the person from the type of the medicine acquired as the sensing information.

The second estimation processor 213 in the fourth embodiment estimates a state of sleep of the person in second estimation processing. The state of sleep includes sleeping time of the person in a predetermined period. The predetermined period is one day, for example. The second estimation processor 213 estimates the sleeping time of the person from the biological information acquired as the sensing information. The second estimation processor 213 estimates the sleeping time from when the person falls asleep to when the person wakes up from the heart rate, the respiratory rate, and the amount of body motion, of the person.

The life support information generator 214 in the fourth embodiment generates life support information based on the state of medicine intake and the state of sleep. The life support information generator 214 determines whether the type of medicine taken by the person is a sleeping medicine. When the type of medicine taken by the person is a sleeping medicine, the life support information generator 214 determines whether sleeping time is shorter than a predetermined time. When determining that the sleeping time is shorter than the predetermined time, the life support information generator 214 generates life support information prompting consultation with a doctor about medication. The predetermined time is seven hours, for example. In contrast, when determining that the sleeping time is equal to or more than the predetermined time, the life support information generator 214 generates life support information indicating that the person is correctly taking the medicine.

For example, when the type of medicine taken by the person is a sleeping medicine and the sleeping medicine is adapted to the person, the sleeping time increases. In contrast, when the type of medicine taken by the person is a sleeping medicine, and the sleeping medicine is not adapted to the person, the sleeping time decreases. Thus, when the type of medicine taken by the person is a sleeping medicine and the sleeping time is shorter than a predetermined time, the life support information generator 214 generates the life support information prompting consultation with a doctor about medication.

The second estimation processor 213 may estimate the number of times of awakening of the person during sleep. The life support information generator 214 may determine whether the number of times of awakening of the person during sleep is more than a predetermined number of times. When the number of times of awakening of the person during sleep is more than the predetermined number of times, the life support information generator 214 may generate the life support information prompting consultation with a doctor about medication. In contrast, when the number of times of awakening during sleep of the person is equal to or less than the predetermined number of times, the life support information generator 214 may generate the life support information indicating that the person is correctly taking the medicine.

For example, the life support information output unit 215 outputs life support information indicating that the person is correctly taking the medicine or life support information prompting consultation with a doctor about medication. The life support information output unit 215 transmits the life support information for supporting a life of the person to the information terminal 3 using the communication unit 23.

For example, when the information terminal 3 receives the life support information prompting consultation with a doctor about medication, a care manager presents the doctor with a type, the amount, and a dosage date and time, of the estimated sleeping medicine, and sleeping time. The life support information includes the type, the amount, the dosage date and time, of the estimated sleeping medicine, and the sleeping time. When the information terminal 3 receives the life support information indicating that the person is correctly taking the medicine, for example, the care manager may or may not present the doctor with the type, the amount, the dosage date and time, of the estimated sleeping medicine, and the sleeping time.

Subsequently, life support information generation processing in the fourth embodiment will be described. Life support processing other than the life support information generation processing in the fourth embodiment is the same as the life support processing in the first embodiment illustrated in FIG. 2.

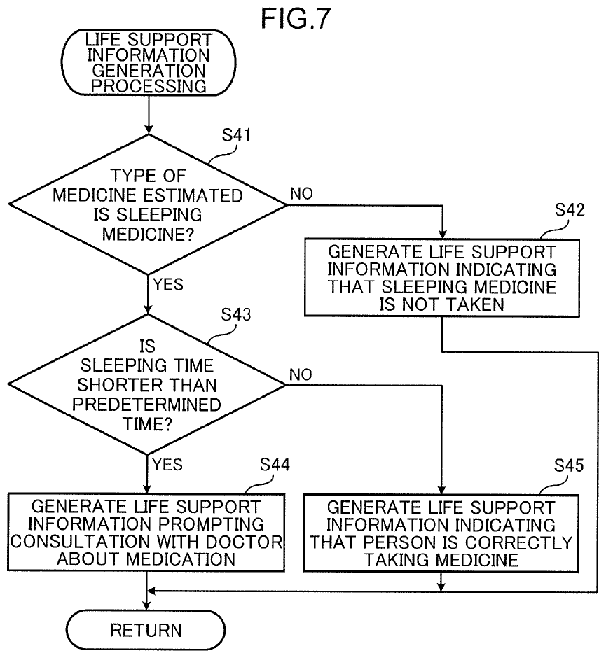

FIG. 7 is a flowchart for illustrating the life support information generation processing in the fourth embodiment.

In step S41, the life support information generator 214 first determines whether the type of medicine estimated by the first estimation processor 212 is a sleeping medicine. Here, when determining that the type of estimated medicine is not a sleeping medicine (NO in step S41), the life support information generator 214 generates life support information indicating that the sleeping medicine is not taken, in step S42.

In contrast, when determining that the type of estimated medicine is a sleeping medicine (YES in step S41), the life support information generator 214 determines whether the sleeping time estimated by the second estimation processor 213 is shorter than the predetermined time, in step S43.

Here, when determining that the estimated sleeping time is shorter than the predetermined time (YES in step S43), the life support information generator 214 generates the life support information prompting consultation with a doctor about medication, in step S44.

In contrast, when determining that the estimated sleeping time is equal to or more than the predetermined time (NO in step S43), the life support information generator 214 generates the life support information indicating that the person is correctly taking the medicine, in step S45.

As described above, the fourth embodiment causes the state of medicine intake of the person and the state of sleep of the person to be estimated, and the life support information to be generated based on the state of medicine intake and the state of sleep, so that the person can be supported to take appropriate medicine.

In each of the above embodiments, each component may be configured by dedicated hardware, or may be implemented by executing a software program suitable for each component. Each component may be implemented by a program execution unit such as a CPU or a processor reading and executing a software program recorded on a recording medium such as a hard disk or a semiconductor memory. Alternatively, the program may be executed by another independent computer system by recording and transferring the program on a recording medium or transferring the program via a network.

Some or all of functions of the devices according to the embodiments of the present disclosure are typically realized as a large scale integration (LSI) that is an integrated circuit. The functions may be individually integrated into one chip, or may be integrated into one chip including some or all of the functions. The integrated circuit is not limited to the LSI, and may be realized by a dedicated circuit or a general-purpose processor. A field programmable gate array (FPGA) that can be programmed after manufacturing an LSI, or a reconfigurable processor that can reconfigure connection and setting of circuit cells inside an LSI, may be used.

Additionally, some or all of functions of the devices according to the embodiments of the present disclosure may be implemented by a processor such as a CPU executing a program.

The numbers used above are merely examples for specifically describing the present disclosure, and the present disclosure is not limited to the illustrated numbers.

The order in which the steps shown in the above flowcharts are performed is merely an example for specifically describing the present disclosure, and any order other than the above may be available as long as a similar effect can be obtained. Some of the above steps may be performed simultaneously (in parallel) with another step.

### INDUSTRIAL APPLICABILITY

The technique according to the present disclosure can sufficiently support life of a person, and thus is useful as a technique for managing health of the person.

1. An information processing method performed by a computer, the method comprising:
acquiring sensing information output from a sensor that senses a state of a person;
executing first estimation processing of estimating a state of substance intake of the person based on the sensing information;
executing second estimation processing of estimating a state of activity of the person, the state being correlated with the state of substance intake, based on the sensing information;
generating life support information on the person based on an execution result of the first estimation processing and an execution result of the second estimation processing; and
outputting the life support information generated.

2. The information processing method according to claim 1, wherein
in execution of the first estimation processing, a state of water intake of the person is estimated,
in execution of the second estimation processing, a state of excretion of the person is estimated, and
in generation of the life support information, the life support information is generated based on balance between the state of water intake and the state of excretion.

3. The information processing method according to claim 2, wherein
the state of water intake includes an amount of water intake of the person in a predetermined period,
the state of excretion includes an amount of urine of the person in the predetermined period, and
in generation of the life support information, the life support information indicating that necessary water is not taken is generated when the amount of water intake is less than the amount of urine in comparison between the amount of water intake and the amount of urine in the predetermined period.

4. The information processing method according to claim 1, wherein
in execution of the first estimation processing, a state of nutrient intake of the person is estimated,
in execution of the second estimation processing, a state of nutrient consumption of the person is estimated, and
in generation of the life support information, the life support information is generated based on balance between the state of nutrient intake and the state of nutrient consumption.

5. The information processing method according to claim 4, wherein
the state of nutrient intake includes the amount of energy intake of the person in a predetermined period,
the state of nutrient consumption includes the amount of energy consumption of the person in the predetermined period,
in generation of the life support information, the life support information indicating that the amount of meal of the person is insufficient is generated when the amount of energy intake is smaller than the amount of energy consumption in comparison between the amount of energy intake and the amount of energy consumption in the predetermined period, and the life support information indicating that the amount of exercise of the person is insufficient is generated when the amount of energy intake is larger than the amount of energy consumption in the comparison.

6. The information processing method according to claim 1, wherein
in execution of the first estimation processing, a state of medicine intake of the person is estimated,
in execution of the second estimation processing, a state of excretion of the person is estimated, and
in generation of the life support information, the life support information is generated based on the state of medicine intake and the state of excretion.

7. The information processing method according to claim 6, wherein
the state of medicine intake includes a type, an amount, and a dosage date and time of medicine taken by the person,
the state of excretion includes properties, an amount, and an excretion date and time of feces excreted by the person,
in generation of the life support information, the life support information prompting consultation with a doctor about medication is generated when it is determined that: the properties of the feces excreted by taking the medicine of the estimated type is not same as the estimated properties of the feces; the amount of the feces excreted by taking the medicine of the estimated type is not same as the estimated amount of the feces; or the feces are not excreted within a predetermined time from the date and time of taking the medicine of the estimated type, and
the life support information includes the type, the amount, and the dosage date and time, of the estimated medicine, and the properties, the amount of excretion, and the excretion date and time of the feces.

8. The information processing method according to claim 6, wherein
the state of medicine intake includes a type, an amount, and a dosage date and time of medicine taken by the person,
the state of excretion includes an excretion date and time and an excretion frequency of urine excreted by the person,
in generation of the life support information, the life support information prompting consultation with a doctor about medication is generated when it is determined that the urine is not excreted within a predetermined time from a date and time when medicine of an estimated type is taken, or when it is determined that an estimated excretion frequency of the urine is higher than a predetermined frequency from the date and time when the medicine of the estimated type is taken, and
the life support information includes the type, the amount, and the dosage date and time, of the estimated medicine, and the excretion date and time, and the excretion frequency, of the urine.

9. The information processing method according to claim 1, wherein
in execution of the first estimation processing, a state of medicine intake of the person is estimated,
in execution of the second estimation processing, a state of sleep of the person is estimated, and
in generation of the life support information, the life support information is generated based on the state of medicine intake and the state of sleep.

10. The information processing method according to claim 9, wherein
the state of medicine intake includes a type, an amount, and a dosage date and time of medicine taken by the person,
the state of sleep includes sleeping time of the person,
in generation of the life support information, the life support information prompting consultation with a doctor about medication is generated when the type of the medicine taken by the person is a sleeping medicine and it is determined that the sleeping time is shorter than a predetermined time after determining whether the sleeping time is shorter than the predetermined time, and
the life support information includes the type, the amount, and the dosage date and time, of the estimated medicine, and the sleeping time.

11. An information processing device comprising:
an acquisition unit that acquires sensing information output from a sensor that senses a state of a person;
a first estimation processing execution unit that executes first estimation processing of estimating a state of substance intake of the person based on the sensing information;
a second estimation processing execution unit that executes second estimation processing of estimating a state of activity of the person, the state being correlated with the state of substance intake, based on the sensing information;
a generator that generates life support information on the person based on an execution result of the first estimation processing and an execution result of the second estimation processing; and
an output unit that outputs the life support information generated.

12. A non-transitory computer readable recording medium storing an information processing program that causes a computer to function so as to:
acquire sensing information output from a sensor that senses a state of a person;
execute first estimation processing of estimating a state of substance intake of the person based on the sensing information;
execute second estimation processing of estimating a state of activity of the person, the state being correlated with the state of substance intake, based on the sensing information;
generate life support information on the person based on an execution result of the first estimation processing and an execution result of the second estimation processing; and
output the life support information generated.
